(19) United States
(12) Patent Application Publication
(10) Pub. No.: US 2004/0180817 A1
(43) Pub. Date: Sep. 16, 2004

(54) Neuropathologies associated with expression of tnf-$g (a)

(76) Inventors: Nicola Ruth Sibson, Reading (GB); Daniel C Anthony, London (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/473,994

(22) PCT Filed: Apr. 5, 2002
(86) PCT No.: PCT/GB02/01633

(30) Foreign Application Priority Data
Apr. 5, 2001 (GB) 0108689.1

(51) Int. Cl. A61K 38/17; G01N 33/53; G01N 33/567

(52) U.S. Cl. 514/12; 435/7.2

(57) ABSTRACT

A treatment for neuropathologies associated with elevated levels of the cytokine TNF-α in the brain is disclosed. The resulting reduction in cerebral perfusion can be eliminated by the administration of an endothelin receptor antagonist, an antagonist to the TNF-α p75 receptor, an endothelin converting enzyme inhibitor or an endothelin neutralising agent. Evaluation of suitable treatment compounds which bind to the TNF-α p75 receptor or the endothelin receptors (ET_{A} and ET_{B}), and which act as an antagonist at these receptors, can be performed using in vivo MRI techniques to detect an increase in cerebral perfusion.

### FIELD OF INVENTION

The present invention relates to the treatment of neuropathologies associated with expression of tumour necrosis factor-α (TNF-α). The present invention further relates to methods of identifying compounds useful in the treatment of these conditions.

### BACKGROUND OF INVENTION

Expression of the proinflammatory cytokine tumour necrosis factor-α (TNF-α) is associated with the pathology of a broad spectrum of central nervous system (CNS) disease and injury. However, the consequences of TNF-α expression—whether detrimental or protective—remains the focus of considerable debate and confusion in the literature.

TNF-α has been quantified in post-mortem tissue from the brains of both cerebral malarial and HIV-1 patients^{2,3}, indicating local production of the cytokine. TNF-α expression has also been demonstrated in post-mortem brain tissue from patients with bacterial meningitis^{1,4}, a condition in which intrathecal levels of TNF-α correlate positively with the degree of blood-brain barrier (BBB) breakdown, disease severity and indices of meningeal inflammation⁵. Furthermore, TNF-α expression is associated with demyelinating multiple sclerosis (MS) lesions⁶ and the presence of TNF-α in cerebrospinal fluid from MS patients correlates with disease activity⁷. Thus, the accumulated evidence suggests a role for TNF-α in the pathophysiology of a variety of CNS disorders, although the mechanisms by which this cytokine contributes to disease or injury severity remain unresolved.

Following both stroke and trauma the inflammatory response has been shown to contribute to secondary injury and increased lesion volume. However, although TNF-α is the archetypal pro-inflammatory cytokine, it can be both neurotoxic and neuroprotective in models of cerebral ischaemia and head injury (for review see ref. 8). It has been suggested that in the early stages of injury over-expression of TNF-α is deleterious, while at later time points it may contribute to recovery of injured tissue^{8,9}. Recently, Gourin and Shackford¹⁰ reported elevated TNF-α levels in cerebral microvascular endothelium isolated from head-injured patients, suggesting possible cerebrovascular effects of this cytokine.

### SUMMARY OF THE INVENTION

Broadly, the present invention is based on the finding that the presence of TNF-α in the brain, and in particular elevated levels of TNF-α, is associated with low cerebral perfusion, which can be eliminated by treatment with an endothelin receptor antagonist. Thus, the present invention proposes the treatment of neuropathologies associated with expression of TNF-α within the brain tissue by the use of (a) endothelin receptor antagonists, (b) endothelin converting enzyme inhibitors, or (c) endothelin neutralising agents. In addition, of the two TNF-α receptor subtypes, p55 and p75, activation of the p75 receptor is required for the TNF-α-induced reduction in perfusion. Thus, the present invention proposes the treatment of neuropathologies in which TNF-α is expressed within the brain tissue by antagonists of the TNF-α p75 receptor-mediated pathway.

Magnetic resonance imaging (MRI) is used clinically for the evaluation of many neuropathologies in which inflammation is implicated. Conventional MRI provides a sensitive measure of tissue structure and water content and, together with intravenous contrast agents, can measure BBB permeability and cerebral perfusion. In addition, diffusion weighted imaging has demonstrated a sensitivity to reversible and irreversible alterations in cellular homeostasis which are undetectable histologically, notably in acute ischaemia and spreading depression¹¹. Owing to the non-invasive nature of MRI, these techniques are ideally suited to the temporal evaluation of brain disease in vivo.

The experiments described herein employed MRI techniques to investigate the effects of a focal striatal injection of TNF-α on cerebral perfusion, on BBB and B-CSF-B viability, and on tissue water diffusion. These experiments demonstrated the diverse actions of TNF-α in the brain and provide a mechanistic basis by which this cytokine may contribute to the pathogenesis of diseases associated with TNF-α expression, such as cerebral malaria, multiple sclerosis, HIV-dementia, cerebral tuberculosis, trypanosomiasis, bacterial meningitis, in which TNF-α is over-expressed within the brain parenchyma. The results reported here identify low cerebral perfusion, compromised neuronal energy metabolism, and damage to the blood brain barriers as effects of elevated TNF-α that may contribute to neuronal degeneration or dysfunction in these diseases.

Using magnetic resonance imaging in vivo the results disclosed herein show that a focal injection of tumour necrosis factor-α into the brain parenchyma induces a rapid reduction in cerebral perfusion and concomitant breakdown of the blood-cerebrospinal fluid barrier. The reduction in cerebral perfusion is completely ameliorated by an endothelin-receptor antagonist. After 24 hours, blood-brain barrier breakdown together with a widespread reduction in tissue water diffusion is evident within the brain parenchyma. This study demonstrates detrimental effects of TNF-α within the deep brain parenchyma, and suggests a therapeutic role for endothelin-receptor antagonists in neuropathologies associated with expression of TNF-α.

Accordingly, in a first aspect, the present invention provides the use of an endothelin receptor antagonist for the preparation of a medicament for the treatment of a neuropathology associated with expression of TNF-α.

In a further aspect, the present invention provides the use of an inhibitor of an enzyme which is capable of catalysing the conversion of endothelin precursors to endothelin peptides for the preparation of a medicament for the treatment of a neuropathology associated with expression of TNF-α.

In a further aspect, the present invention provides the use of an endothelin neutralising agent for the preparation of a medicament for the treatment of a neuropathology associated with expression of TNF-α.

In a further aspect, the present invention provides the use of an antagonist to the TNF-α p75 receptor and/or pathway for the preparation of a medicament for the treatment of a neuropathology associated with expression of TNF-α.

Examples of conditions which are neuropathologies associated with expression of TNF-α include (i) cerebral malaria, (ii) multiple sclerosis, (iii) HIV-dementia, (iv) cerebral tuberculosis, (v) trypanosomiasis or (vi) bacterial meningitis. The present invention is applicable to both the therapeutic and prophylactic treatment of these conditions. For example, prophylactic treatment might be particularly useful in the case of malaria.

In a further aspect, the present invention provides a method of treating a neuropathology associated with expression of TNF-α, the method comprising administering to a patient in need of therapeutically or prophylactically effective amount of (a) an endothelin receptor antagonist, (b) an inhibitor of an enzyme which is capable of catalysing the conversion of big endothelins to their mature forms, (c) an endothelin neutralising agent, and/or (d) an antagonist to the TNF-α p75 receptor and/or pathway.

In a further aspect, the present invention provides a method of identifying compounds useful for the treatment of a TNF-α mediated neuropathology, the method comprising contacting one or more candidate compounds and (a) a TNF-α p75 receptor or (b) an endothelin receptor (ET_{A} and/or ET_{B}) and identifying the compounds which bind to the either the TNF-α p75 receptor or the endothelin receptor (ET_{A} and/or ET_{B}).

The method may then comprise the additional step of determining whether the compound is a receptor antagonist, e.g. has the property of blocking the action of TNF-α at either the p75 receptor or downstream, including at the endothelin receptors, and testing it, e.g. in vivo using the MRI techniques disclosed herein, to determine whether the compound is capable of increasing cerebral perfusion reduced by the TNF-α mediated pathway disclosed herein.

Embodiments of the present invention will now be described by way of example and not limitation with reference to the accompanying figures.

### BRIEF DESCRIPTION OF THE FIGURES

FIG. 1: Time course of injected/non-injected striatal rCBV ratios. Graph showing effect of a focal striatal injection of either TNF-α or vehicle on rCBV. Values are expressed as ratios of rCBV in the treated (left) striatum vs. the untreated (right) striatum. Data are presented for three groups of animals: (i) control, intrastriatal injection of vehicle only (black bars); (ii) intrastriatal injection of 0.3 μg recombinant rat (rr) TNF-α (grey bars); and (iii) intrastriatal injection of 1.5 μg rrTNF-α (hatched bars). Values close to 1.0 indicate no change in striatal perfusion, as seen in control animals. All values are mean±S.D. Asterisks indicate a significant difference between control and treated groups: *P<0.05, **P<0.02, ***P<0.001.

FIG. 2: Striatal rCBV ratios demonstrating the effect of an endothelin receptor antagonist. Graph showing effect of the ET receptor antagonist Ro 46-2005 on the rrTNF-α-induced rCBV changes 1.5 h after intrastriatal injection. Values are expressed as ratios of rCBV in the treated (left) striatum vs. the untreated (right) striatum. Data are presented for five groups of animals: (i) control, intrastriatal injection of vehicle only (n=4); (ii) intrastriatal injection of 0.3 μg rrTNF-α (n=6); (iii) intrastriatal injection of 1.5 μg rrTNF-α (n=3); (iv) intravenous injection of Ro 46-2005+intrastriatal injection of 1.5 μg rrTNF-α (n=6); and (v) intravenous injection of sterile water+intrastriatal injection of 1.5 μg rrTNF-α (n=4). Values close to 1.0 indicate no change in striatal perfusion, and all values are mean±S.D. *P<0.005, unpaired t test. No significant differences were found either between the control and 1.5 μg rrTNF-α +Ro 46-2005 groups, or between the 1.5 μg rrTNF-α and 1.5 μg rrTNF-α +H₂O groups.

FIG. 3: Striatal rCBV ratios demonstrating the effect of rhuTNF-α in comparison to rrTNF-α and an endothelin receptor antagonist. Graph showing effect of the rhuTNF-α on rCBV. Values are expressed as ratios of rCBV in the treated (left) vs. the untreated (right) striatum. Data are presented for five groups of animals: (i) control, intrastriatal injection of vehicle only (n=4); (ii) intrastriatal injection of 0.3 μg rrTNF-α (n=6); (iii) intrastriatal injection of 1.5 μg rrTNF-α (n=3); (iv) intrastriatal injection of 0.3 μg rhuTNF-α (n=5) and (v) intrastriatal injection of 1.5 μg rhuTNF-α (n=5). Values close to 1.0 indicate no change in striatal perfusion, and all values are mean±S.D. *P<0.005, unpaired t test. No significant differences were observed between the control, 0.3 μg rhuTNF-α and 1.5 μg rhuTNF-α groups.

### DETAILED DESCRIPTION

Definitions

In the present invention, an “endothelin receptor antagonist” is a substance that interferes with the action of endothelin peptides at an endothelin receptor.

Such substances may act by (a) binding to the receptor, or (b) otherwise inhibiting it from binding or interacting with an endothelin peptide. Examples of such substances include ETA antagonists such as BQ-123, BMS-182874, LU1135252, EMD94246, FR139317 or PD156707; ETB antagonists such as RES-701-1, BQ-788 or BQ2020; or combined ETA/ETB antagonists such as TAK-044, Bosentan, Ro 46-2005 or IRL3630A; or combinations of these substances.

In the present invention, an “endothelin converting enzyme inhibitor” is a substance that inhibits the conversion of endothelin precursors to endothelin peptides. These substances include endothelin converting enzyme (ECE-1 & ECE-2) inhibitors such as Halistand Disulfate B. This is described in Kedzierski & Yanagisawa, Ann. Rev. Pharmacol. Toxicol., 41:851-876, 2001, which also describes endothelin receptors and other materials and method useful in carrying out the present invention, such as the receptors and converting enzymes mentioned herein.

In the present invention, an “endothelin neutralising agent” is a substance that binds to the endothelin peptides and effectively inactivates them, for instance a specific binding partner such as an antibody, and more preferably a neutralising antibody. Techniques for screening for endothelin peptide specific binding partners and producing antibodies capable of binding to and inactivating an endothelin peptide are well known in the art. Methods of producing antibodies include immunising a mammal (e.g. mouse, rat, rabbit, horse, goat, sheep or monkey) with an endothelin peptide or a fragment thereof. Antibodies may be obtained from immunised animals using any of a variety of techniques known in the art, and screened, preferably using the binding of the antibody to an endothelin peptide of interest and/or to determine whether the antibody is a neutralising antibody, that is it is capable of binding to and inactivating an endothelin peptide or inhibiting or preventing its interaction with a receptor. For instance, Western blotting techniques or immunoprecipitation may be used (Armitage et al, Nature, 357:80-82, 1992). Isolation of antibodies and/or antibody-producing cells from an animal may be accompanied by a step of sacrificing the animal.

As an alternative or supplement to immunising a mammal with an endothelin peptide, an antibody specific for the protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

The antibodies may be modified in a number of ways that are well known in the art. Indeed the term “antibody” should be construed as covering any binding substance having a binding domain with the required specificity. Thus, the present invention includes the use of antibody fragments, derivatives, functional equivalents and homologues of antibodies, including synthetic molecules and molecules whose shape mimics that of an antibody enabling it to bind an antigen or epitope. Humanised antibodies in which CDRs from a non-human source are grafted onto human framework regions, typically with the alteration of some of the framework amino acid residues, to provide antibodies which are less immunogenic than the parent non-human antibodies, are also included within the present invention.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2 188 638 A or EP 0 239 400 A. Cloning and expression of chimeric antibodies are described in EP 0 120 694 A and EP 0 125 023 A.

Hybridomas capable of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

Methods of Screening

As described above, the present invention provides methods of screening for compounds which are capable of reversing a TNF-α associated reduction in cerebral perfusion and which may therefore be useful in the treatment of the neuropathologies which are the subject of the invention.

Accordingly, the present invention provides a means to screen compounds that are likely to reverse TNF-α-mediated pathology in the brain. In particular the invention enables the screening of (a) substances that are capable of binding to the endothelin receptors and inhibiting the binding of TNF-α-induced endothelin with its receptors, (b) substances that are able to inhibit the conversion of TNF-α-induced endothelin precursors to mature endothelin peptides (ECE-1 & ECE-2 inhibitors), (c) substances that are able to block the binding of TNF-α to the TNF-α p75 receptor.

For example, in a further aspect, the present invention provides a method of identifying compounds useful for the treatment of a TNF-α associated neuropathology, the method comprising contacting one or more candidate compounds and the TNF-α p75 receptor or the endothelin receptors (ET_{A} and/or ET_{B}) and identifying the compounds which bind to the either the TNF-α p75 receptor or the endothelin receptors (ET_{A} and/or ET_{B}).

The method may then comprise the additional step of determining whether the compound is an endothelin receptor or TNF-α p75 receptor antagonist, e.g. has the property of blocking the action of TNF-α at either the p75 receptor or downstream at the endothelin receptors, and testing it, e.g. in vivo using the MRI techniques disclosed herein, to determine whether the compound is capable of increasing cerebral perfusion reduced by the TNF-α mediated pathway disclosed herein.

TNF-α binds to two transmembrane receptors of approximately 55 (p55, TNFR1, CD120a) and 75 kDa (p75, TNFR2, CD120b) (Aggarwal and Natarajan, 1996, Eur.

Cytokine Network 7:93-124). While the p55 TNF-α receptor is ubiquitously expressed, the p75 receptor is predominantly expressed by haematopoietic and endothelial cells. These receptors have no previously described consensus sequence involved in signal transduction and show no homology in their intracellular domains, which suggests that they activate distinct signalling pathways and mediate distinct cellular processes. The recombinant rat TNF-α (rrTNF-α) used in the studies described above binds non-specifically to both TNF-α receptor subtypes, whilst the recombinant human TNF-α (rhuTNF-α) will only bind to the p55 receptor in rat brain (Lewis et al., 1991, Proc. Natl. Acad. Sic. USA 88: 2830-2834; Stefferl et al., 1996, Br. J. Pharmacol. 118:1919-1924). Thus, we used rhuTNF-α to identify the receptor subtype involved in the TNF-α induced reduction in perfusion. In these experiments, intracerebral injection of rhuTNF-α caused no reduction in cerebral perfusion, in contrast to intracerebral rrTNF-α injection (as described above). These data show that activation of the p75 TNF-α receptor, either alone or in combination with the p55 receptor, is required for the observed reduction in cerebral perfusion. Consequently, antagonists of the p75 TNF-α receptor subtype represents a route of therapeutic intervention in neuropathologies associated with TNF-α expression within the brain.

In carrying out these methods, it may be convenient to screen a plurality of candidate compounds, e.g. as present in a library, at the same time, e.g. by contacting a mixture of different candidate compounds with the interacting peptides, and then in the event of a positive result resolving which member of the mixture is active. These technique are used in high throughput screening (HTS) to increase the numbers of compounds, e.g. resulting from combinatorial chemistry program or present in library derived from a natural source material, which can be screened in a method.

The precise format of the assays of the invention may be varied by those of skill in the art using routine skill and knowledge. For example, interaction between substances may be studied in vitro by labelling one with a detectable label and bringing it into contact with the other which has been immobilised on a solid support. Suitable detectable labels, especially for peptidyl substances include ³⁵S-methionine which may be incorporated into recombinantly produced peptides and polypeptides. Recombinantly produced peptides and polypeptides may also be expressed as a fusion protein containing an epitope which can be labelled with an antibody. Fusions can also be used to display the peptides or receptors, e.g. in a protein such as thioredoxin, in order to present the peptide motifs in a correct three dimensional structure. The substance which is immobilized on a solid support may be immobilized using an antibody against that protein bound to a solid support or via other technologies which are known per se. A preferred in vitro interaction may utilise a fusion protein including glutathione-S-transferase (GST). This may be immobilized on glutathione agarose beads. In an in vitro assay format of the type described above a test compound can be assayed by determining its ability to diminish the amount of labelled peptide or polypeptide which binds to the immobilized GST-fusion polypeptide. This may be determined by fractionating the glutathione-agarose beads by SDS-polyacrylamide gel electrophoresis. Alternatively, the beads may be rinsed to remove unbound protein and the amount of protein which has bound can be determined by counting the amount of label present in, for example, a suitable scintillation counter.

The amount of candidate substance or compound which may be added to an assay of the invention will normally be determined by trial and error depending upon the type of compound used. Typically, from about 0.01 to 100 nM concentrations of putative inhibitor compound may be used, for example from 0.1 to 10 nM. Greater concentrations may be used when a peptide is the test substance.

Compounds which may be used may be natural or synthetic chemical compounds used in drug screening programmes. Extracts of plants which contain several characterised or uncharacterised components may also be used.

Pharmaceutical Uses

The substances of the invention can be used in the treatment neuropathologies associated with expression of TNF-α, and in particular, (i) cerebral malaria, (ii) multiple sclerosis, (iii) HIV-dementia, (iv) cerebral tuberculosis, (v) trypanosomiasis and (vi) bacterial meningitis. The composition may be administered alone or in combination with other treatments for these conditions, either simultaneously or sequentially dependent upon the condition to be treated.

Whether it is a polypeptide, antibody, peptide, nucleic acid molecule, small molecule, mimetic or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practioners and other medical doctors.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, lactated Ringer's injection. Preservatives, stabilisers, buffers, antioxidants and/or other additives may be included, as required. Examples of techniques and protocols mentioned above can be found in Remington's Pharmaceutical Sciences, 16th edition, Osol, A. (ed), 1980.

Materials and Methods

Animal Preparation

Adult male Wistar rats (Harlan-Olac, UK) were anaesthetised with fentanyl/fluanisone and midazolam (0.68 ml/kg of each). Using a 50 μm-tipped glass pipette, 1 μl rat recombinant TNF-α (NIBSC, Potters Bar, UK) solution was injected stereotaxically 1 mm anterior and 3 mm lateral to Bregma, at a depth of 4 mm into the left striatum. Animals were injected with either 0.3 μg/μl or 1.5 μg/μl of TNF-A, each in 0.1% BSA in low-endotoxin saline, or with vehicle solution only. Animals were positioned in the MRI probe (3.4 cm i.d. Alderman-Grant resonator) using a bite-bar. During MRI, anaesthesia was maintained with 0.8-1.2% halothane in 50% N₂O/50% O₂, ECG was monitored non-invasively and body temperature was maintained at −37° C. All procedures were approved by the United Kingdom Home Office.

Magnetic Resonance Imaging

Magnetic resonance images were acquired using a 300 MHz Varian Inova spectrometer (Varian, Palo Alto, Calif.). Anatomical images were acquired using a T₂-weighted sequence (repetition time, TR, 3 sec; echo time, TE, 80 msec). Diffusion weighted images were acquired with a pulsed-gradient spin-echo sequence (TR 1.0 sec; TE 40 msec), using diffusion weighting values of 125, 750 and 1500 s.mm⁻², a diffusion time of 20 msec and a diffusion gradient duration of 10 msec. Diffusion gradients were applied separately along three orthogonal axes and apparent diffusion coefficient (ADC) “trace” maps were calculated⁴⁰. Navigator echoes were used for motion correction⁴¹. Perfusion maps were generated as described previously¹² from 40 time-series images during which 150 ml of a gadolinium-based contrast agent (Omniscan, Nycomed Amersham, UK) was injected via a tail vein, over a 4 sec period from image 8. Spin-echo T₁ weighted images (TR 500 msec; TE 20 msec) were acquired both pre- and 10 minutes post-contrast agent injection to look for image enhancement owing to BBB/B-CSF-B permeability. Slice thickness was 1 mm for coronal images and 2 mm for horizontal images, except for the perfusion data sets, which were all 1 mm.

Experimental Protocol

Four studies were carried out to investigate different aspects of the brain response to TNF-α.

(a) Acute Effects of TNF-α on Cerebral Perfusion and B-CSF-B/BBB Viability

Three groups of animals were used: (i) control, vehicle only (n=4); (ii) 0.3 μg TNF-α (n=6); and (iii) 1.5 μg TNF-α (n=4). Pre-contrast T₁-weighted images, perfusion data and post-contrast T₁-weighted images were acquired at 1, 2, 3 and 5 h post TNF-α injection in the coronal plane, and at 1.5, 2.5, 3.5 and 5.5 h in the horizontal plane.

(b) Acute Effects of TNF-α on Tissue Water Diffusion

Two groups of animals were used: (i) control, vehicle only (n=4); and (ii) 0.3 μg TNF-α (n=7). Diffusion weighted images were acquired each hour (1-6 h) after TNF-α injection in the coronal plane, and at the half-hour time points (1.5-6.5 h), together with T₂-weighted images, in the horizontal plane.

(c) Chronic Effects of TNF-α

In studies (a) and (b) all animals recovered from anaesthesia after the final acquisition and were re-imaged using all MRI protocols at either 24 h (control, n=4; 0.3 μg TNF-α, n=8; 1.5 μg TNF-α, n=3) or 72 h (control, n=3; 0.3 μg TNF-α, n=6) after stereotaxic injection. Following MRI at 24 or 72 h, the brains were perfusion-fixed for histological and immunocytochemical analysis.

(d) Effect of an Endothelin Receptor Antagonists on Acute Cerebral Perfusion Changes

Two groups of animals were used: (i) intravenous injection of the ET receptor antagonist Ro 46-2005 (1 mg in 0.25 ml sterile water) 10 min before injection of 1.5 μg TNF-α (n=6); and (ii) intravenous injection of sterile water (0.25 ml) 10 min before injection of 1.5 μg TNF-α (n=4). MRI data was acquired as for (a) in the horizontal plane at 1.5 h only.

Histological Analysis

Following MRI, all animals were deeply anaesthetised and transcardially perfused with heparinised saline and periodate lysine paraformaldehyde (PLP). Brains were post-fixed for 4 h in PLP, immersed in 30% sucrose buffer for 24 h and then embedded in Tissue Tek (Miles Inc, Elkhart) at −40° C. Cresyl violet-stained sections (50 μm) were examined for neuronal damage.

Immunohistochemistry on 10 μm cresyl violet-counter-stained sections was used to confirm the presence and distribution of leukocyte populations. Antigens were detected using a three-step indirect method⁴². Macrophage or neutrophil infiltration was quantified by counting the number of ED1-positive⁴³ or HB199-positive⁴⁴ cells, respectively. Leukocyte numbers were calculated as an average per mm² in three non-overlapping fields containing the highest density of recruited cells within the parenchyma.

MRI Data Analysis

Regions of Interest (ROI) encompassing the striatum were defined on T₂-weighted images in each hemisphere, and applied to all images or calculated data maps for quantitation. For the rCBV maps and T₂-weighted images the data are expressed as a ratio of injected/non-injected striatal values. All values are mean±S.D. All ROI and statistical analysis was performed on images obtained in the horizontal plane (at the level of the injection site), and coronal plane data was used for qualitative purposes only.

Since data were not acquired at every time point from all animals over the acute time course (for technical reasons), a mixed-effect model followed by pair-wise t tests⁴⁵ was used to determine any statistical differences between the rCBV time courses for each group. Unpaired or paired t tests were used to determine significant differences at 24 and 72 h for all MRI parameters.

Results

A minimally invasive technique to focally microinject TNF-α or vehicle into the brain parenchyma was used. Consequently, in the vehicle-injected animals, no visible leukocyte recruitment or damage to the brain parenchyma was observed at any time point. On T₂-weighted scout images at 1 hour the injection bolus was visible as a small hyperintense area in the left striatum in all animals, and subsequent scans were positioned directly through the injection site.

Acute Effects of TNF-α on Cerebral Perfusion

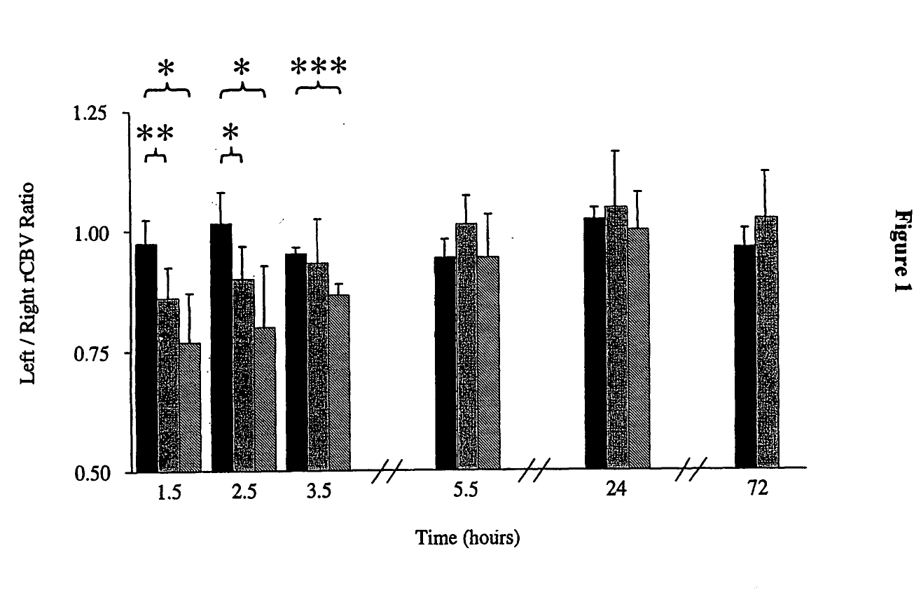

An acute reduction in local cerebral perfusion in the injected striatum at 1.5 h as a consequence of rrTNF-α injection into the brain was observed, which returned gradually to normal by ˜5.5 h. The local changes in cerebral perfusion were assessed by calculating the ratio of regional Cerebral Blood Volume (rCBV) within a Region of Interest (ROI) in the injected striatum versus a matched area in the non-injected striatum of the same animal. In animals injected with either 0.3 μg or 1.5 μg of recombinant rat TNF-α, the ratio of injected/non-injected striatal rCBV was significantly reduced compared to the vehicle-injected group at 1.5 h (unpaired t tests; low dose P<0.02, high dose P<0.05; FIG. 1). This reduction in rCBV was dose-dependent, with a greater reduction at the higher dose (˜23%) than at the lower dose (˜14%) as compared to vehicle-injected animals. Although the statistical data analysis was performed on the images acquired at 1.5, 2.5, 3.5 and 5.5 h, the reduction in perfusion was observed from as early as 1 h after the rrTNF-α injection in coronal images. The rCBV changes occurred prior to leukocyte recruitment to the brain parenchyma, which was first evident 4 h after the injection of rrTNF-α. At this time, a small number of recruited monocytes were visible in cuffs around the penetrating vessels (50.8±5.0 per mm² ED1-stained cells).

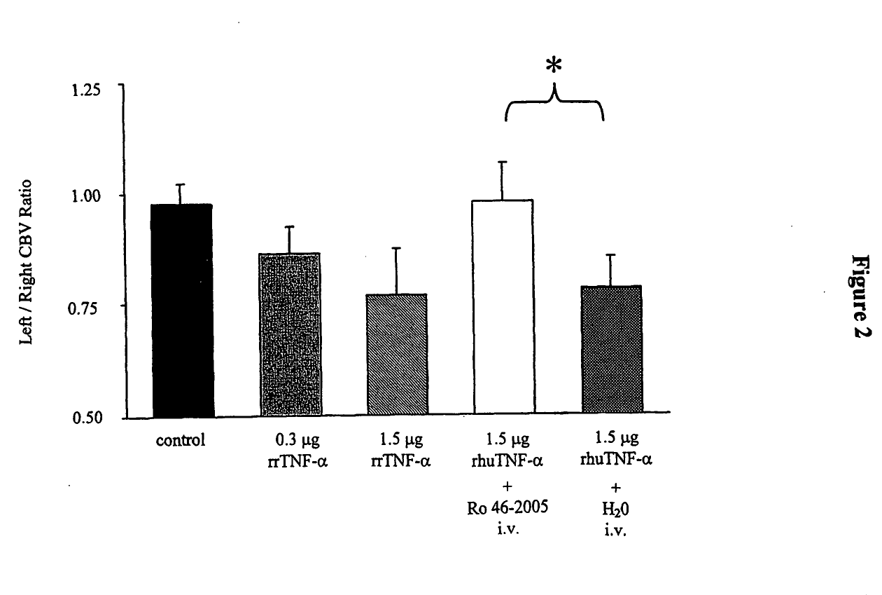

The reduction in rCBV at 1.5 h in the injected striatum was eliminated by intravenous injection of the endothelin (ET) receptor antagonist Ro 46-2005 (5 mg/kg) 10 minutes prior to intracerebral rrTNF-α (1.5 μg) injection (FIG. 2). In control animals injected intravenously with the vehicle (sterile water) 10 minutes prior to intracerebral rrTNF-α (1.5 μg), the reduction in striatal rCBV was still evident, and comparable to the initial group of animals injected with 1.5 μg rrTNF-α (FIG. 2). The difference in injected/non-injected striatal rCBV ratios for the two groups receiving an intravenous injection (Ro 46-2005 or vehicle) prior to intracerebral rrTNF-α injection was highly significant (unpaired t test, P<0.005).

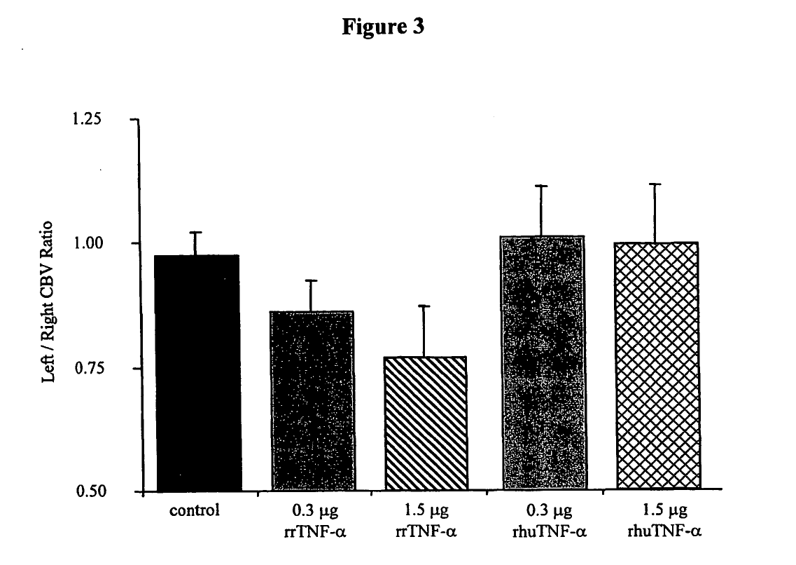

No reduction in rCBV in the injected striatum was observed in response to intracerebral injection of rhuTNF-α (0.3 μg and 1.5 μg) in comparison to vehicle treated animals (FIG. 3), indicating that activation of TNFR1 alone does not result in a reduction in rCBV.

Acute Effects of TNF-α on B-CSF-B and BBB Integrity From as early as 1.5 h after injection of 1.5 μg TNF-α (2-3 h with 0.3 μg TNF-α), enhancement of the meninges on post-contrast T₁-weighted images was observed. This enhancement indicates breakdown of the blood-cerebrospinal fluid barrier (B-CSF-B) and was first evident in the meninges overlying the parietal cortex. The breakdown of the B-CSF-B was not detected by histochemical localisation of the tracer horseradish peroxidase (HRP) at this time point, and preceded recruitment of any inflammatory cells to the meninges. Pre-treatment with Ro 46-2005 did not significantly alter the effect of TNF-α on the B-CSF-B at 1.5 h.

Over subsequent hours the B-CSF-B breakdown spread to encompass meningeal layers surrounding the frontal cortex. By 5.5 h the B-CSF-B breakdown was just visible histologically using HRP, and marked monocyte-restricted recruitment to the meninges occurred from ˜4 h. In some cases, the MRI signal enhancement appeared to have spread into the outermost cortical layers by 5.5 h, suggesting compromise of the pial and cortex-penetrating vessels. In the coronal plane, meningeal enhancement around the entire injected hemisphere was observed, and this was often particularly clear around the piriform cortex where we found large numbers of monocytes histologically.

Acute Effects of TNF-α on Tissue Water Diffusion

From 1 to 4 h, small increases in the tissue water diffusion at the injection site were observed in all animals, which corresponded, spatially, to regions of T₂ hyperintensity. This acute increase in both T₂ signal intensity (5-13%) and diffusion (6-8%) reflects the small increase in extracellular water arising from the injection bolus, and resolved as the fluid was cleared.

Chronic Effects of TNF-α on Tissue Water Diffusion, B-CSF-B/BBB Integrity, and Cerebral Perfusion

Although ELISA measurements show that all TNF-α has been cleared from the brain parenchyma by 24 h, tissue water diffusion in the injected striatum of TNF-α injected animals was found to be significantly reduced (paired t test, P<0.02, 0.3 μg TNF-α group) compared with the non-injected striatum at 24 h (Table 1). Despite the focal nature of the cytokine injection, the reduction in tissue water diffusion was not restricted to the striatum and also encompassed surrounding cortical regions. The reduction in tissue water diffusion observed in the TNF-α-injected animals was not dose dependent, with similar reductions in both groups (Table 1). There were no significant differences between the injected and non-injected hemispheres in the control animals. The reduction in ADC was not affected by pre-treatment with the ET-receptor antagonist Ro 46-2005, with a significant difference (paired t test, P<0.03) between the injected and non-injected striatal values being evident (Table 1). Similarly, there was a significant difference between the injected and non-injected striatal ADC values in the animals injected with 1.5 μg rhuTNF-α (paired t test, P<0.02; Table 1). However, although a reduction in ADC was apparent in the injected hemisphere in 3 out of 5 animals injected with the lower dose (0.3 μg) of rhuTNF-α, this did not reach significance (paired t test, P=0.136).

Breakdown of the B-CSF-B in all animals injected with rrTNF-α and rhuTNF-α persisted to 24 h, when large numbers of monocytes were present in the meninges. Low-level breakdown of the BBB in the brain parenchyma was also observed 24 h after rrTNF-α injection on post-contrast T₁-weighted images. This breakdown was more evident with the higher dose (1.5 μg) of rrTNF-α (increase in signal intensity of injected striatum post-gd vs. pre-gd=8.1+2.1%), but less apparent than that observed previously following intrastriatal IL-1β injection (Blamire et al., 2000). The pattern of BBB breakdown was similar to the tissue water diffusion changes at 24 h, encompassing both striatal and cortical regions. At this time point recruitment of monocytes into the brain parenchyma was marked (189±7 per mm² with 0.3 μg rrTNF-α; n=3). Pre-treatment with the ET-receptor antagonist Ro 46-2005 did not significantly affect the level of BBB breakdown observed in animals injected with 1.5 g TNF-α (increase in signal intensity of injected striatum=10.3±2.7%). However, in animals injected with 1.5 μg rhuTNF-α the degree of BBB breakdown appeared to be substantially reduced (increase in signal intensity of injected striatum=5.2±1.6%), which may be related to a lower level of monocyte recruitment (95±33 per mm²; n=3) compared to that induced by rrTNF-α at this time point.

72 h after TNF-α injection, both the BBB and B-CSF-B were intact, and no significant differences in tissue water diffusion was found, T₂ intensity or rCBV between the injected and non-injected hemispheres in any animals. However, the number of ED-1 positive macrophages present within the brain parenchyma was maximal (361±79 per mm² with 0.3 μg TNF-α) at this time. There was no apparent neuronal cell death at any time point following the single bolus injections of rrTNF-α or rhuTNF-α, as evidenced by cresyl violet staining.

Discussion

In this study we have shown that a focal, intrastriatal injection of TNF-α in the rat brain results in (i) an acute, dose-dependent reduction in cerebral blood volume that is mediated by endothelin, and coupled to activation of the TNF-α receptor 2 (TNFR2) pathway, (ii) early breakdown of the blood-CSF barrier and delayed breakdown of the blood-brain barrier, and (iii) a delayed reduction in tissue water diffusion. At all times leukocyte recruitment to the brain (parenchyma and meninges) was restricted solely to monocytes, as reported previously^{46,47}. These results are in contrast to our previous findings following intrastriatal injection of IL-1β, which induced an increase, rather than a decrease, in cerebral blood volume and recruited only neutrophils to the brain parenchyma¹². In peripheral tissues, IL-1β and TNF-α have been reported to have similar effects and, it is surprising, therefore, that these cytokines have different effects within the CNS. Despite these differences, both cytokines result in a decrease in tissue water diffusion, although this is delayed in TNF-α-injected animals compared to IL-1β-injected animals. The implications of the current findings are discussed below.

Effects of TNF-α on Cerebral Blood Volume

Our data demonstrate that there is a profound, acute reduction in striatal rCBV as a direct consequence of focal rrTNF-α injection. Few investigations of the effects of TNF-α on cerebral perfusion have been reported previously, and where data is available the results are somewhat contradictory. Several years ago, Megyeri et al.¹³ demonstrated vasoconstriction in pial arterioles following intracisternal injection of rhuTNF-α into newborn piglets. In contrast, Brian and Faraci¹⁴ recently demonstrated dilation of pial arterioles following superfusion of the rat cortex with TNF-α. Both of these studies report the effects of TNF-α on the superficial pial arterioles of the brain, rather than the intraparenchymal microvasculature. Similarly, intracisternal injections of TNF-α have been shown to decrease whole brain CBF in rabbits¹⁵ and to increase cortical blood flow in rats¹⁶. Again, it is likely that in both studies the effects of TNF-α were exerted on the superficial, rather than intraparenchymal, vessels, and that the differences reflect either species or dose differences. In rat models of cerebral ischaemia, inhibition of endogenous TNF-α has been shown to improve microvascular perfusion¹⁷ and enhance cerebral blood flow during reperfusion¹⁸. On this basis, it has been suggested that expression of TNF-α following focal cerebral ischaemia may contribute to impairment of microvascular perfusion, either as a consequence of recruited leukocytes obstructing cerebral vessels or via a direct vasoconstrictor effect of the cytokine itself¹⁷. Our data demonstrate clearly that an intracerebral injection of rrTNF-α causes acute, temporary vasoconstriction of local parenchymal vessels that is independent of recruited leukocytes.

Since the reduction in rCBV precedes monocyte recruitment, we hypothesised that this might occur via TNF-α-induced expression of endothelin peptides (ET-1 and ET-3), which are known vasoconstrictors. Many pathologies associated with increased cytokine production also exhibit elevated levels of circulating ET-1, and peripheral injection of TNF-α into rats significantly increases plasma ET-1 concentrations within 15 minutes¹⁹. Our data demonstrate that the vasoconstrictor effects of rrTNF-α within the brain parenchyma in vivo can be completely eliminated by prior administration of an ET receptor antagonist which blocks both ET_{A} and ET_{B} receptors²². We suggest, therefore, that the observed effects of rrTNF-α on rCBV are mediated via the action of ET on its receptors. ET-1 production by both bovine and human cerebral endothelial cells in culture is increased by TNF-α^{20,21}, and vascular smooth muscle cells have also been shown to secrete ET-1 in inflammatory lesions⁴⁸. It is likely, therefore, that the observed reduction in rCBV is caused by the action of TNF-α on the brain microvessel endothelial and smooth muscle cells to provoke the release of ET, which subsequently causes vasoconstriction primarily through its action on the smooth muscle cell ET_{A} receptors⁴⁸.

TNF-α binds to two transmembrane receptors of approximately 55 (p55, TNFR1) and 75 kDa (p75, TNFR2)⁴⁹. While the TNFR1 is ubiquitously expressed, the TNFR2 is predominantly expressed by haematopoietic and endothelial cells, and they are thought to activate distinct signalling pathways and mediate distinct cellular processes. The rrTNF-α used in these studies binds non-specifically to both TNF-α receptor subtypes, whilst rhuTNF-α will bind only to TNFR1 in rat brain^{50,51}. In contrast to rrTNF-α injection, intrastriatal injection of rhuTNF-α caused no reduction in rCBV. These data indicate that activation of the TNFR2, either alone or in combination with the TNFR1, is essential for the TNF-α-induced reduction in rCBV, and that activation of this pathway leads to ET release, probably via activation of the microvascular endothelial cells. These data may explain the discrepancies in TNF-α-mediated effects on cerebral perfusion reported previously, where rhuTNF-α was used in rodent studies before rrTNF-α became widely available.

In this study we have used a single bolus injection of TNF-α into the striatum. Previously, we have demonstrated by ELISA that following a bolus injection of TNF-α the level of immunoreactive TNF-α in the brain parenchyma has fallen to 50% of maximum after 4 h and is no longer quantifiable by 24 h⁵². As a result, effects of TNF-α may be short-lived under this experimental protocol in comparison to neurological conditions in which TNF-α is expressed chronically. Therefore, whilst no histopathology or neuronal loss was observed in this study, chronic TNF-α expression may result in a prolonged perfusion deficit that is deleterious to neuronal viability. In experimental stroke models it is well documented that a reduction of 80-90% in cerebral perfusion of short duration invariably results in energy failure and neuronal death^{23,24}. However, much less severe reductions in cerebral perfusion, if prolonged, can also lead to neuronal death²⁵. Therefore, in neurological conditions where TNF-α expression is prolonged, this may cause a long-term perfusion deficit that is detrimental to neuronal viability.

Both cerebral malaria^{53,1} and the Plasmodium berghei ANKA model of cerebral malaria⁵⁴ are associated with high levels of cerebral TNF-α expression, adhesion of monocytes to the cerebral vasculature, and increased permeability of the BBB—which are all features associated with the single bolus injection of TNF-α into the brain parenchyma. Further, a significant increase in the expression of TNFR2, but not TNFR1, has been found on brain microvessels during cerebral malaria in susceptible mice, and mice deficient in TNFR2 (but not those deficient in TNFR1) are significantly protected from experimental cerebral malaria⁵⁵. Thus, the effects of TNF-α on rCBV, mediated via the TNFR2 pathway and ET production, may be a contributing factor to neuronal dysfunction or degeneration in cerebral malaria, in which the cause of neuronal damage, and ultimately patient death, are still unknown. In addition, MS pathology is associated with significant axonal degeneration²⁶, which occurs by mechanisms that remain unclear. However, ischaemia in axons has been shown to lead to the reversal of the Na⁺/Ca²⁺ exchanger, influx of Ca²⁺, and, consequently, axonal degeneration²⁷. Thus, chronic low rCBV induced by TNF-CL within MS plaques may result in metabolic insufficiency and axonal degeneration.

Effects of TNF-α on B-CSF-B and BBB Integrity

TNF-α is thought to play a role in BBB disruption associated with brain injury²⁸ and bacterial meningitis, and in vitro has been shown to decrease the trans-endothelial resistance in cerebrovascular-derived endothelial cells²⁹. However, few studies have considered variations in BBB compromise between the different CNS compartments. In the current study, the early unilateral increase in B-CSF-B permeability (as distinct from BBB permeability) preceded leukocyte recruitment to the brain. This effect on the B-CSF-B may reflect direct actions of TNF-α on the vasculature, as studies with tracers (Sibson and Anthony, unpublished data) indicate that a bolus of fluid (as injected in this study) will diffuse fairly rapidly out of the striatum and alongside the major cortical vessels, to reach the meninges within 1.5-2 h. Furthermore, the data indicate that the B-CSF-B breakdown is leukocyte-independent, since it preceded macrophage recruitment to the meninges and was no longer apparent at 72 h when recruited macrophages were numerous. Although monocytes can cross an intact BBB and B-CSF-B, breakdown of these barriers may facilitate presentation of chemokines and thus recruitment to the meninges.

In contrast, breakdown of the BBB within the brain parenchyma at 24 h was coincident with significant macrophage recruitment to the parenchyma. This finding differs from our previous studies of BBB viability using HRP, in which only very minimal leakage of tracer, localised specifically to the larger parenchymal vessels, was observed 24 h after a single bolus intraparenchymal injection of TNF-α⁴⁶. In addition, the B-CSF-B breakdown observed at the early time points was visible by contrast-enhanced MRI before it became detectable using HRP. The current data suggest, therefore, that contrast-enhanced MRI measurements offer a more sensitive method of detecting BBB/B-CSF-B permeability than the HRP method, probably owing to the considerably smaller molecular weight of the gadolinium-based agent (0.57 kDa) compared to HRP (40 kDa).

Pre-treatment with the non-specific ET receptor antagonist Ro 46-2005 had no effect on the changes in BBB and B-CSF-B permeability, suggesting that these events are not mediated by the TNF-α-induced ET pathway responsible for the rCBV reduction. However, the ET system is widespread in the brain, with ET_{A}, ET_{B}, ET-1 and ET-3 being expressed by vascular, neuronal and glial cells⁴⁸. Given that the Ro 46-2005 injection was administered intravenously, it is possible that it may not antagonise non-vascular effects of ET occurring deep within the brain parenchyma, such as the observed BBB breakdown and ADC reduction. It is unlikely, however, that the B-CSF-B changes would persist if they were mediated by the ET system. Rosenberg et al⁵⁶ have previously demonstrated a dose-dependent parallel increase in capillary permeability and expression of proteolytic enzymes 24 h after intracerebral infusion of TNF-α, which could be blocked by an inhibitor of matrix metalloproteinases. On this basis, they suggest that TNF-α may modulate delayed capillary permeability via the matrix metalloproteinase gelatinase B. Interestingly, there appeared to be a reduction in the degree of BBB permeability at 24 h following rhuTNF-α injection compared with the rrTNF-α-injected animals. These data suggest that both receptor pathways contribute to the processes underlying the BBB breakdown.

Effects of TNF-α on Tissue Water Diffusion

The areas of reduced tissue water ADC observed at 24 h corresponded to the regions of BBB breakdown, and indicate a relatively widespread effect of the focal cytokine injection. Again, this is likely to result from spread of the injected bolus to neighbouring cortical regions. Reduced tissue water diffusion has been extensively documented in acute brain ischaemia⁵⁷, although the exact mechanisms responsible for these changes remain unclear. In ischaemia, the temporal evolution of reduced diffusion appears to follow the loss of high-energy metabolites³⁰ and is thought to reflect compromise of tissue energy metabolism. It has been suggested that the mechanism for the reduction in diffusion may be a shift of tissue water from the faster diffusing extracellular space to the more slowly diffusing intracellular space^{30,31}, as a result of energetic failure, disruption of cell membrane potentials and redistribution of ions. However, there is also evidence that reduced overall tissue water diffusion represents changes in absolute diffusibility in all brain compartments^{32,33}. Observations of a transient reduction in ADC during spreading depression³⁴ indicate that changes in tissue water diffusion are linked to disruption of tissue energy homeostasis, rather than ischaemia per se. This hypothesis is supported by our previous finding that IL-1β causes a reduction in ADC that is accompanied by an increase in rCBV and no indicators of ischaemia ². In the current study rCBV was found to be normal within the areas of reduced ADC at 24 h, again suggesting that ischaemia is unlikely to be the cause of these changes.

As with the BBB permeability changes, pre-treatment with the non-specific ET receptor antagonist Ro 46-2005 had no effect on the observed ADC reduction, although as discussed above this does not necessarily preclude the ET system from playing a role in these changes. However, in animals injected intrastriatally with rhuTNF-α there appeared to be a dose-dependent effect on tissue ADC. This finding suggests that, as for the BBB permeability changes, the pathways induced by both TNF-α receptors may be involved in the processes underlying the ADC changes. It has been shown that TNF-α is not directly toxic to neurones^{58,59}. However, recent studies have shown that TNF-α markedly inhibits glutamate uptake in both human and rat astrocytes in culture³⁵⁻³⁷. Thus, glutamate-induced toxicity and resultant energetic compromise of neurones may contribute to the observed reduction in tissue water diffusion at 24 h. Alternatively, it has been suggested that TNF-α may impair the ability of astrocytes to provide adequate energy substrates to neurones for oxidation³, which also could result in neuronal dysfunction.

Our single bolus injections of TNF-α resulted in no overt neuronal cell death, despite significant, but reversible, MRI-visible changes. Thus, reversible TNF-α-induced decreases in cerebral perfusion and compromise of neuronal energy metabolism may provide an explanation for one of the puzzling clinical sequelae of cerebral malaria—sudden losses of consciousness, sometimes with rapid recovery and no evidence of neuronal cell death. Furthermore, the adenovirus experiments suggest that prolonged TNF-α expression in the brain parenchyma may be profoundly detrimental to neuronal function and survival. Our data suggest that both endothelin receptors, and the TNFR2 pathway, are potential targets for therapeutic intervention in neuropathologies, such as cerebral malaria, that are associated with high cerebral TNF-α expression.

TABLE 1
Apparent diffusion coefficients of tissue water in each striatum.
Apparent Diffusion Coefficient (×10⁻⁴ mm²/sec)

| | Vehicle | | 0.3 μg rrTNF-α | | 1.5 μg rrTNF-α | |
|---|---|---|---|---|---|---|
| Time | Left | Right | Left | Right | Left | Right |
| 24 h | 6.78 ± 0.35 | 6.79 ± 0.35 | 6.28^{a} ± 0.38 | 6.99 ± 0.46 | 6.30^{a} ± 0.52 | 6.90 ± 0.35 |
| 72 h | 6.59 ± 0.25 | 6.50 ± 0.22 | 6.82 ± 0.42 | 6.55 ± 0.36 | | |
| | Ro 46-2005 + | | | | | |
| | 1.5 μg rrTNF-α | | 0.3 μg rhuTNF-α | | 1.5 μg rhuTNF-α | |
| Time | Left | Right | Left | Right | Left | Right |
| 24 h | 6.72^{b} ± 0.25 | 7.23 ± 0.17 | 7.15 ± 0.28 | 7.33 ± 0.26 | 6.76^{a} ± 0.20 | 7.14 ± 0.31 |
| 72 h | | | | | | |
| Maps of the apparent diffusion coefficient (ADC) of tissue water were calculated from the diffusion-weighted images, and the ADC was measured within a region of interest in each striatum (Left = treated, Right = control). Values are mean ± S.D. for n = 4 (control groups), n = 8 (0.3 μg rrTNF-α, | | | | | | |
| # 24 h), n = 6 (0.3 μg rrTNF-α, 72 h), n = 4 (1.5 μg rrTNF-α, 24 h), n = 4 (Ro 46-2005 + 1.5 μg rrTNF-α, 24 h), n = 5 (0.3 μg rhuTNF-α, 24 h) and n = 5 (1.5 μg rhuTNF-α, 24 h). Significant differences from control (right) striatum were determined by paired tests, | | | | | | |

^{a}P < 0.02,
^{b}P < 0.05.

### REFERENCES

The references cited herein are all expressly incorporated by reference.

1. Brown et al. Cytokine expression in the brain in human cerebral malaria. J Infect Dis 180, 1742-6 (1999).

2. Achim et al. Quantitation of human immunodeficiency virus, immune activation factors and quinolinic acid in AIDS brains. Journal of Clinical Investigation 91, 2769-2775 (1993).

3. Nuovo et al. In situ detection of polymerase chain reaction-amplified HIV-1 nucleic acids and tumor necrosis factor-alpha RNA in the central nervous system. Am J Pathol 144, 659-66 (1994).

4. Waage et al. Local production of tumor necrosis factor alpha, interleukin 1, and interleukin 6 in meningococcal meningitis. Relation to the inflammatory response. J Exp Med 170, 1859-67 (1989).

5. Sharief et al. Blood-brain barrier damage in patients with bacterial meningitis: association with tumor necrosis factor-alpha but not interleukin-1 beta. J Infect Dis 166, 350-8 (1992).

6. Woodroofe et al. Cytokine mRNA expression in inflammatory multiple sclerosis lesions: detection by non-radioactive in situ hybridization. Cytokine 5, 583-8 (1993).

7. Hauser et al. Cytokine accumulations in CSF of multiple sclerosis patients: frequent detection of interleukin-1 and tumor necrosis factor but not interleukin-6. Neurology 40, 1735-9 (1990).

8. Shohami et al. Dual role of tumor necrosis factor alpha in brain injury. Cytokine Growth Factor Rev 10, 119-30 (1999).

9. Scherbel et al. Differential acute and chronic responses of tumor necrosis factor-deficient mice to experimental brain injury. Proc Natl Acad Sci USA 96, 8721-6 (1999).

10. Gourin & Shackford, Production of tumor necrosis factor-alpha and interleukin-1beta by human cerebral microvascular endothelium after percussive trauma. J Trauma 42, 1101-7 (1997).

11. Hoehn-Berlage, Diffusion-weighted NMR imaging: application to experimental focal cerebral ischemia. NMR Biomed 8, 345-58 (1995).

12. Blamire et al. Interleukin-1beta-induced changes in blood-brain barrier permeability, apparent diffusion coefficient, and cerebral blood volume in the rat brain: A magnetic resonance study. J Neurosci 20, 8153-9 (2000).

13. Megyeri et al. Recombinant human tumor necrosis factor alpha constricts pial arterioles and increases blood-brain barrier permeability in newborn piglets. Neurosci Lett 148, 137-40 (1992).

14. Brian, Jr. & Faraci, Tumor necrosis factor-alpha-induced dilatation of cerebral arterioles. Stroke 29, 509-15 (1998).

15. Tureen. Effect of recombinant human tumor necrosis factor-alpha on cerebral oxygen uptake, cerebrospinal fluid lactate, and cerebral blood flow in the rabbit: role of nitric oxide. J Clin Invest 95, 1086-91 (1995).

16. Angstwurm et al. Tumour necrosis factor alpha induces only minor inflammatory changes in the central nervous system, but augments experimental meningitis. Neuroscience 86, 627-34 (1998).

17. Dawson et al. Inhibition of tumor necrosis factor-alpha reduces focal cerebral ischemic injury in the spontaneously hypertensive rat. Neurosci Lett 218, 41-4 (1996).

18. Lavine et al. Circulating antibody against tumor necrosis factor-alpha protects rat brain from reperfusion injury. J Cereb Blood Flow Metab 18, 52-8 (1998).

19. Klemm et al. Endothelin 1 mediates ex vivo coronary vasoconstriction caused by exogenous and endogenous cytokines. Proc Natl Acad Sci USA 92, 2691-5 (1995).

20. Durieu-Trautmann, et al. Nitric oxide and endothelin secretion by brain microvessel endothelial cells: regulation by cyclic nucleotides. J Cell Physiol 155, 104-11 (1993).

21. Skopal et al. Regulation of endothelin release from human brain microvessel endothelial cells. J Cardiovasc Pharmacol 31, S370-2 (1998).

22. Clozel et al. Pathophysiological role of endothelin revealed by the first orally active endothelin receptor antagonist. Nature 365, 759-61 (1993).

23. Tamura et al. Focal cerebral ischaemia in the rat: 2. Regional cerebral blood flow determined by [14C]iodoantipyrine autoradiography following middle cerebral artery occlusion. J Cereb Blood Flow Metab 1, 61-9 (1981).

24. Bolander et al. Regional cerebral blood flow and histopathologic changes after middle cerebral artery occlusion in rats. Stroke 20, 930-7 (1989).

25. Jacewicz et al. The CBF threshold and dynamics for focal cerebral infarction in spontaneously hypertensive rats. J Cereb Blood Flow Metab 12, 359-70 (1992).

26. Ferguson et al. Axonal damage in acute multiple sclerosis lesions. Brain 120, 393-9 (1997).

27. Waxman et al. Non-synaptic mechanisms of Ca(2+)-mediated injury in CNS white matter. Trends Neurosci 14, 461-8 (1991).

28. Feuerstein et al. Cytokines, inflammation, and brain injury: role of tumor necrosis factor-alpha. Cerebrovasc Brain Metab Rev 6, 341-60 (1994).

29. de Vries et al. The influence of cytokines on the integrity of the blood-brain barrier in vitro. J Neuroimmunol 64, 37-43 (1996).

30. van der Toorn et al. Dynamic changes in water ADC, energy metabolism, extracellular space volume, and tortuosity in neonatal rat brain during global ischemia. Magn Reson Med 36, 52-60 (1996).

31. Van Zijl et al. Complete separation of intracellular and extracellular information in NMR spectra of perfused cells by diffusion-weighted spectroscopy. Proc Natl Acad Sci USA 88, 3228-32 (1991).

32. van der Toorn et al. Diffusion of metabolites in normal and ischemic rat brain measured by localized 1H MRS. Magn Reson Med 36, 914-22 (1996).

33. Duong et al. Evaluation of extra- and intracellular apparent diffusion in normal and globally ischemic rat brain via 19F NMR. Magn Reson Med 40, 1-13 (1998).

34. Latour et al. Spreading waves of decreased diffusion coefficient after cortical stimulation in the rat brain. Magn Reson Med 32, 189-98 (1994).

35. Fine et al. Tumor necrosis factor alpha inhibits glutamate uptake by primary human astrocytes. Implications for pathogenesis of HIV-1 dementia. J Biol Chem 271, 15303-6 (1996).

36. Hu et al. Cytokine effects on glutamate uptake by human astrocytes. Neuroimmunomodulation 7, 153-9 (2000).

37. Ye et al. Cytokine modulation of glial glutamate uptake: a possible involvement of nitric oxide. Neuroreport 7, 2181-5 (1996).

38. Yu et al. Tumor necrosis factor-alpha and interleukin-1 alpha enhance glucose utilization by astrocytes: involvement of phospholipase A2. Mol Pharmacol 48, 550-8 (1995).

39. Magistretti et al. Energy on demand. Science 283, 496-7 (1999).

40. Basser et al. MR diffusion tensor spectroscopy and imaging. Biophys J 66, 259-67 (1994).

41. Ordidge et al. Correction of motional artifacts in diffusion-weighted MR images using navigator echoes. Magn Reson Imaging 12, 455-60 (1994).

42. Hsu et al. The use of antiavidin antibody and avidin-biotin-peroxidase complex in immunoperoxidase technics. Am J Clin Pathol 75, 816-21 (1981).

43. Dijkstra et al. The heterogeneity of mononuclear phagocytes in lymphoid organs: distinct macrophage subpopulations in the rat recognized by monoclonal antibodies ED1, ED2 and ED3. Immunology 54, 589-99 (1985).

44. Anthony et al. Matrix metalloproteinase expression in an experimentally-induecd DTH model of multiple sclerosis in the rats CNS. J Neuro 87, 62-72 (1998).

45. Vonesh et al. Linear and Nonlinear Models for the Analysis of Repeated Measurements, (Marcel Dekker Inc, New York, 1997).

46. Anthony D C, Bolton S J, Fearn S, Perry V H. Age-related effects of interleukin-1B on polymorphonuclear neutrophil-dependent increases in blood-brain barrier permeability in rats. Brain 1997; 120: 435-444.

47. Glabinski A, Krajewski S, Rafalowska J. Tumor necrosis factor-alpha induced pathology in the rat brain: characterization of stereotaxic injection model. Folia Neuropathol 1998; 36: 52-62.

48. Kedzierski R M, Yanagisawa M. Endothelin system: the double-edged sword in health and disease. Annu Rev Pharmacol Toxicol 2001; 41: 851-876.

49. Aggarwal B B, Natarajan K. Tumor necrosis factors: developments during the last decade. Eur Cytokine Netw 1996; 7: 93-124.

50. Lewis M, Tartaglia L A, Lee A, Bennett G L, Rice G C, Wong G H, et al. Cloning and expression of cDNAs for two distinct murine tumor necrosis factor receptors demonstrate one receptor is species specific. Proc Natl Acad Sci USA 1991; 88: 2830-2834.

51. Stefferl A, Hopkins S J, Rothwell N J, Luheshi G N. The role of TNF-alpha in fever: opposing actions of human and murine TNF-alpha and interactions with IL-beta in the rat. Br J Pharmacol 1996; 118: 1919-1924.

52. Anthony D, Blond D, Dempster R, Perry V. Castellano-Lopez B and Nieto-Sampedro M, editors. Progress in Brain Research. Elsevier Science B.V., 2001: Chapter 40.

53. Kwiatkowski D, Hill A V S, Sambou I, Twumasi P, Castracane J, Manogue K R, et al. TNF concentration in fatal cerebral, non-fatal cerebral, and uncomplicated plasmodium falciparum malaria. Lancet 1990; 336: 1201-1204.

54. Neill A L, Hunt N H. Effects of endotoxin and dexamethasone on cerebral malaria in mice. Parasitology 1995; 111: 443-454.

55. Lucas R, Juillard P, Decoster E, Redard M, Burger D, Donati Y, et al. Crucial role of tumor necrosis factor (TNF) receptor 2 and membrane-bound TNF in experimental cerebral malaria. Eur J Immunol 1997; 27: 1719-1725.

56. Rosenberg G A, Estrada E Y, Dencoff J E, Stetler-Stevenson W G. Tumor necrosis factor-alpha-induced gelatinase B causes delayed opening of the blood-brain barrier: an expanded therapeutic window. Brain Res 1995; 703: 151-155.

57. Hoehn-Berlage M. Diffusion-weighted NMR imaging: application to experimental focal cerebral ischemia. NMR Biomed 1995; 8: 345-358.

58. Piani D, Spranger M, Frei K, Schaffner A, Fontana A. Macrophage-induced cytotoxicity of N-methyl-D-aspartate receptor positive neurons involves excitatory amino acids rather than reactive oxygen intermediates and cytokines. Eur J Immunol 1992; 22: 2429-2436.

59. Barone F C, Arvin B, White R F, Miller A, Webb C L, Willette R N, et al. Tumor necrosis factor-alpha. A mediator of focal ischemic brain injury. Stroke 1997; 28: 1233-1244.

1. Use of (a) an endothelin receptor antagonist, (b) an antagonist to the TNF-α p75 receptor, (c) an endothelin converting enzyme inhibitor, or (d) an endothelin neutralising agent for the preparation of a medicament for the treatment of a neuropathology associated with the expression of TNF-α.

2. The use of claim 1, wherein the neuropathologies associated with expression of TNF-α include: cerebral malaria, multiple sclerosis, HIV-dementia, cerebral tuberculosis, trypanosomiasis and bacterial meningitis.

3. The use of claims 1 and 2, wherein the medicament is administered prophylactically.

4. The use of any one of the preceding claims, wherein the medicament is administered therapeutically.

5. A method of identifying compounds useful for the treatment of a TNF-α mediated neuropathology, the method comprising:
contacting one or more candidate compounds and (a) a TNF-α p75 receptor or (b) an endothelin receptor (ET_{A} and or ET_{B}); and
identifying the compounds which bind to either the TNF-α p75 receptor or the endothelin receptor (ET_{A} and or ET_{B}).

6. The method of claim 5, further comprising:
determining whether the compound is a receptor antagonist.

7. The method of claim 6, wherein the step of determining whether the compound is a receptor antagonist comprises determining whether it has the property of blocking the action of TNF-α at either the p75 receptor or downstream including at the endothelin receptors.

8. The method of claim 6, wherein testing the compound involves the use of in vivo MRI techniques to determine whether the compound is capable of increasing cerebral perfusion reduced by the TNF-α mediated pathway.
